(19) United States
(12) Patent Application Publication
(10) Pub. No.: US 2006/0057221 A1
(43) Pub. Date: Mar. 16, 2006

(54) Polymeric microparticulates for sustained release of drug and their preparation methods

(76) Inventors: Hyeok Lee, Seongnam-si (KR); Ham Yong Park, Seoul (KR); Jeong Hwa Yang, Seongnam-si (KR); Jung Ju Kim, Yongin-si (KR)

Correspondence Address: JONES DAY, 222 EAST 41ST ST, NEW YORK, NY, 10017, US

(21) Appl. No.: 10/534,991

(22) PCT Filed: Nov. 12, 2003
(86) PCT No.: PCT/KR03/02437

§ 371 Date: May 12, 2005

(30) Foreign Application Priority Data
Nov. 13, 2002 (KR) 10-2002-0070317

(51) Int. Cl. A61K 9/50; A61K 9/16; B01J 13/04; B01J 13/02

(52) U.S. Cl. 424/490; 264/4.1

(57) ABSTRACT

The present invention relates to polymeric microparticulates for sustained release of drug and to the process for the preparation thereof. The process of the present invention for preparing polymeric microparticulates based on microcoagulation phenomenon of water-soluble polymer not only improves loading amount of drug but also minimizes initial burst of drug, thereby providing polymeric microparticulates enabling sustained and prolonged release of drug

### TECHNICAL FIELD

The present invention relates to polymeric microparticulates for sustained release of drug and to the process for preparing them.

### BACKGROUND ART

As processes for preparing microparticulates for drug delivery using polymer, solvent evaporation method (N. Wakiyama et al., Chem. Pharm. Bull., 30(7), 2621-2628, 1982), solvent extraction method (J. M. Ruiz et al., Int. J. Pharm., 49, 69-77, 1989), phase separation method (N. Nihant et al., J. Controlled Release, 35, 117-125, 1995), coacervation method (J. C. Leroux et al., Int. Symp. Control. Rel. Bioact. Mater., Controlled Release Society, Inc., 21 #1118, 1994), salting out method (B. Gander et al., J. Microencapsulation, 12(1), 83-97, 1995) and spray drying method (R. Arshady et al., Polym. Eng. Sci., 30(15), 915-924, 1990) can be enumerated. As final characteristics of microparticulates such as particular size, loading amount of drug and release property of drug are largely affected by the preparation methods, adequate method should be selected by considering not only properties of polymer and drug but also desired physical properties of microparticulates.

Among the methods mentioned above, solvent evaporation method and solvent extraction method based on multiple emulsion have been intensively studied, and those are known as general methods for preparing microparticulates using polyester polymers. Such methods for preparing polymeric microparticulates using multiple emulsion method have advantage of easily obtaining microparticulates. However, in case water-soluble drug is used, since the drug diffuses toward external continuous phase during the process of preparing microparticulates, loading efficiency of drug seriously decreases, and thus, the amount of the drug distributed on the surface of microparticulates increases, resulting in initial burst of drug.

On the other hand, Korean Patent Laid-open No. 2002-0005215 discloses methods of encapsulating a protein drug within polyester polymeric microparticulates using reversible microcoagulation phenomenon of the protein drug within solvent mixture of dichloromethane and ethylacetate. According to said method, sustained release of the protein drug has been achieved, and initial burst of the drug has been inhibited. However, the method could be applied to only limited cases based on unique properties of protein drugs, and in case of drugs other than protein drugs, problems such as lowered loading efficiency of drug and initial burst of drug still remained.

In addition, Korean Patent Laid-open No. 1997-069033 describes methods for preparing microparticulates using multiple emulsion method of solidifying polymeric microparticulates in a short period by adding in advance ethylacetate that dose not dissolve polymer but miscible with water, to external continuous phase. According to said method, loading efficiency of drug increases due to the reduction of time in preparing microparticulates. Yet, said method could be applied only to low molecular weight drugs whose water solubility is at least 500 mg/ml, and it resulted in the increase of loading amount of drug but still showed the problem of initial burst of drug to over 60%. Additionally, even though drug is in salt form or hydrophilic, if its water solubility is very low, i.e. about 10 mg/ml, the volume of internal water phase is limited at the time of preparing W/O type primary emulsion and thus amount of drug introduced must also be limited. Therefore, the amount of drug released from microparticulates is likely to be very little so that the amount would be insufficient for providing therapeutic effect. If the amount of drug over saturation concentration to internal water phase is used, polymeric microparticulates could not be prepared via multiple emulsion method.

U.S. Pat. Nos. 6,419,961, 5,585,460 and 4,652,441 disclose methods for preparing copolymeric (poly(lactic acid-co-glycolic acid)) microparticulates including peptide drug such as leuprorelin acetate using multiple emulsion method. In particular, U.S. Pat. No. 4,652,441 increased viscosity of internal water phase by introducing water-soluble polymer such as gelatin, albumin, pectin and agar along with drug, leading to double encapsulation of gelatin and poly(lactic acid-co-glycolic acid), thereby obtaining injectable formulation for prolonged release. However, said method have disadvantage of complicated preparation procedure, that is, in case of using gelatin to increase viscosity of internal water phase, heating to high temperature, 80° C. is required to allow even distribution of drug within primary emulsion, and cooling to 20-30° C. is required at the time of re-dispersing the primary emulsion in external continuous phase. Further, said preparation method has limitation in that it could only be applied to drugs having heat stability.

The inventors of the present invention intended to resolve the problems occurring at the time of preparing polymeric microparticulates based on multiple emulsion process, i.e. low loading amount and initial burst of drug.

The object of the present invention lies in providing polymeric microparticulates enabling sustained release of drug and their preparation methods.

### DISCLOSURE OF THE INVENTION

The present invention relates to polymeric microparticulates for sustained release of drug and to their preparation methods.

The polymeric microparticulates of the present invention is prepared by a method comprising (1) adding secondary organic solvent into primary organic solvent containing biodegradable polymer and hydrophobic surfactant to prepare polymer solution; (2) dissolving and/or dispersing drug(s) in aqueous solution including water-soluble polymer and hydrophilic surfactant, and then adding the solution to the polymer solution prepared in said step (1) to prepare primary emulsion solution (water-in-oil (W/O)), where microcoagulated particles of the water-soluble polymer is formed by dehydration of internal water phase of the primary emulsion, leading to encapsulation of the drug into said microparticulates; and (3) dispersing said primary emulsion into external continuous phase to solidify the polymeric microparticulates. Alternatively, said polymeric microparticulates can be obtained by further conducting conventional filtration and washing procedure in the step (3).

In the below, the process for preparing polymeric microparticulates of the present invention will be explained in detail with respect to each steps.

Step 1: Preparation of Polymeric Solution

First, polymer solution is prepared by adding secondary organic solvent into primary organic solvent containing biodegradable polymer and hydrophobic surfactant.

As the biodegradable polymers, polyester polymer can be used, and preferably, at least one selected from the group consisting of poly(lactic acid) (PLA), poly(glycolic acid) (PGA), poly(lactic acid-co-glycolic acid) (PLGA) and polycaprolactone (PCL) can be used. Said polymers are known as polymers with excellent biocompatibility and biodegradability since it is decomposed into harmless chemicals, i.e. water and carbon dioxide through citric acid cycle which is one of ordinary metabolic process of body (S. J. Holland et al., J. Controlled Release, 4, 155-180, 1986). The biodegradable polymer is not particularly limited but preferably ones with molecular weight in a range of 5,000 to 210,000 are used. Also, the biodegradable polymer can be added to 10 to 60% (w/v) of the organic solvent within the polymer solution.

In addition, the present invention provides the method of preparing polymeric microparticulates by further adding crystalline polymer in said step (1). The crystalline polymer acts as a drug release modifier. As the crystalline polymer, any injectable biocompatible material can be used without particular limitation, yet preferably, poly(ethylene glycol) (PEG) or poly(lactic acid), more preferably, poly(ethylene glycol) is used. Low molecular weight PEG is known as a biocompatible polymer clinically used for intraarticular injection. Preferred molecular weight of PEG is 200 to 5,000. In case the molecular weight of PEG is less than 200, PEG is not formed into crystals, thus fails to act as a drug release modifier, whereas in case molecular weight exceeds 5000, it could not be excreted via kidney (K. K. Huang, T. W. Chang and T. W. Tzeng, Int. J. Pharm., 156, 9-15, 1997). Mass ratio between the crystalline polymer and biodegradable polymer is 0.1:99.9 to 20:80, preferably, 1:99 to 10:90.

In particular, in case said biodegradable polymer whose mole fraction between poly(lactic acid) and poly(glycolic acid) is 50:50 is a low molecular weight copolymer, as it is an amorphous polymer in a rubbery state, the formation of pores and water channels which are main release pathway of drug encapsulated in microparticulates, is inhibited, thus overall release rate of drug tends to be too low. In such case, use of poly(ethylene glycol) as a drug release modifier in physical combination with amorphous polymer facilitates the formation of pores and water channel via formation of crystal area within the amorphous rubbery polymeric microparticulates, leading to easy control of drug release.

On the other hand, as a said hydrophobic surfactant, at least one selected from the group consisting of fatty acid, olefin, alkyl carbon, silicone, sulfate ester, fatty alcohol sulfate, sulfated fat and oil, sulfonic acid salt, aliphatic sulfonate, alkylaryl sulfonate, ligminsulfonate, phosphoric acid ester, polyoxyethylene, polyglycerol, polyol, imidazoline, alkanolamine, hetamine, sulfomethamine, phosphatide and sorbitan fatty acid ester can be used, and preferably, sorbitan fatty acid ester, more preferably, sorbitan trioleate can be used. The hydrophobic surfactant can be added to 0.1 to 30% (v/v) of organic solvent within polymer solution, preferably, 5 to 20% (v/v).

Said primary organic solvent is required to have miscibility with biodegradable polymer and hydrophobic surfactant, and phase separation against water. Said primary organic solvent is not particularly limited, provided that it satisfies the above requirements, yet, at least one selected from dichloromethane, chloroform, cyclohexane and ethylacetate can be used.

Said secondary organic solvent should be miscible with the primary organic solvent, and biodegradable polymer and hydrophobic surfactant contained in the solvent, and is also required to be miscible with water. Said secondary organic solvent is not particularly limited, provided that it satisfies the above requirements, yet, at least one selected from acetone, acetonitrile, dimethylsulfoxide, tetrahydrofuran and dioxan can be used.

In the present invention, it is preferred to use solvent mixture of the primary organic solvent in which biodegradable polymer and hydrophobic surfactant included, and secondary organic solvent satisfying water miscibility. Solvent mixture of dichloromethane and acetone is more preferred. Volume ratio between the primary organic solvent and secondary organic solvent is 95:5 to 50:50, preferably 75:25 to 55:45. Total volume of the primary organic solvent and secondary organic solvent is 1/500 to 1/100 based on the volume of external continuous phase, for example, aqueous polyvinylalcohol solution, preferably, 1/400 to 1/200.

Step 2: Preparation of Primary Emulsion and Primary Encapsulation of Drug Via the Formation of Microcoagulated Particles of Water-Soluble Polymer

The amount of drug over saturated concentration was dissolved and dispersed in aqueous solution containing water-soluble polymer and hydrophilic surfactant, and the mixture was added to the polymer solution prepared in the step 1 and vigorously stirred to prepare primary emulsion (water-in-oil (W/O)). At this time, miscibility with water and the secondary organic solvent within mixed solvent containing biodegradable polymer and hydrophobic surfactant leads to rapid dehydration of internal water phase. Thus, solubility of the water-soluble polymer rapidly decreases to form particles of extremely small size, and in this procedure, the drug is firstly encapsulated into said microparticulates of water-soluble polymer. Therefore, the internal water phase of the primary emulsion prepared in this step exists as a state where microparticulates of the water-soluble polymer in which the drug was encapsulated is dispersed.

The water-soluble polymer used in the present invention is a material with excellent biocompatibility, harmless to a living body and when dissolved in water, exhibits high viscosity. As a said water-soluble polymer, at least one selected from the group consisting of cellulose, hemicellulose, pectin, lignin, starch of storage carbohydrate, chitosan, xanthan gum, alginic acid, pullulan, curdlan, dextran, levan, hyaluronic acid, glucan, collagen and salts thereof can be used, and it is preferred to use hyaluronic acid or its salt. Viscosity of said water-soluble polymer in the aqueous solution before dehydration is 300 to 50,000 cp (centi-poise), preferably, 500 to 30,000 cp.

In addition, at this step 2 of the present invention, for the preparation of polymeric microparticulates, other components can be further added to increase water solubility of the water-soluble polymer. If the water-soluble polymer is chitosan, it is preferred to conduct dissolving chitosan in aqueous solution of organic acid such as formic acid, citric acid, acetic acid and lactic acid and inorganic acid such as hydrochloric acid. At this time, concentration of acid to water is preferred to be 0.5 to 3.0% (w/v).

The hydrophilic surfactant is used for evenly dispersing the amount of drug over saturated concentration. As the hydrophilic surfactant, at least one selected from the group consisting of protein surfactant such as bovine serum albumin (BSA) or carbopol, polyoxyethylene-polyoxypropylene block copolymer and polyoxyethylene sorbitan fatty acid ester (Tween series), preferably, polyoxyethylene sorbitan fatty acid ester surfactant is used, more preferably, polyoxyethylene sorbitan monooleate (product name: Tween 80) is used. The hydrophilic surfactant is added to 0.1 to 30% (w/w) of water, preferably, 1 to 20% (w/w).

Examples of applicable drug in the present invention have no special limitation. For example, as bisphosphonate drugs, etidronate, clodronate, pamidronate, alendronate, ibandronate, risedronate, zolendronate, tiludronate, YH 529, icadronate, olpadronate, neridronate, EB-1053 and salts thereof can be used. Said drug is preferred to have water solubility of 0.1 μg/ml to 1000 mg/ml, preferably, 10 mg/ml to 500 mg/ml.

On the other hand, volume ratio between internal water phase and organic phase is 1:5 to 1:30, preferably, 1:10 to 1:20.

Step 3: Step of Preparing Polymeric Microparticulates Via Solidifying by Dispersing Primary Emulsion in External Continuous Phase

As external continuous phase for dispersing primary emulsion, aqueous solution of sodium dodecyl sulphate (SDS), cetyltrimethyl ammonium bromide (CTAB), methyl cellulose (MC), gelatin, polyoxyethylene sorbitan monooleate or polyvinyl alcohol (PVA) can be used, and preferably, aqueous polyvinyl alcohol solution can be used. If aqueous polyvinyl alcohol solution is used, the concentration of polyvinyl alcohol is 0.1 to 5% (w/v), preferably, 0.3 to 2% (w/v). Molecular weight of polyvinyl alcohol is 10,000 to 100,000, preferably, 13,000 to 23,000, and its degree of hydrolysis is 75 to 95%, preferably, 83 to 89%. Additionally, other ingredients, for example, ethyl acetate conventionally added in the preparation of multiple emulsion can be added in said continuous phase. In such case, ethyl acetate is added to 1-20% (v/v) of PVA aqueous solution, preferably 5-10% (v/v).

The polymeric microparticulates prepared according to the present invention have an average diameter of particle of 0.1 to 200 μm, preferably, 10 to 100 μM, and are characterized in that they can be administered via syringe needle through intravenous, subcutaneous or intramuscular route. Further, said microparticulates are spherical particles in which enormous pores and water channels are formed, and since they have larger surface area compared to film- or cylindrical preparations having same weight controlled release of drug is achieved.

Microcoagulated particles of water-soluble polymer are distributed in the pores existing inside of the polymeric microparticulates prepared according to the present invention, and the drug is encapsulated within the water-soluble polymeric microparticulates. As a result, an effect of double encapsulation of drug within water-soluble polymer and biodegradable polymer is achieved. Based on the double encapsulation, loss of drug toward external continuous phase in the preparation process of microparticulates can be minimized, and initial burst of drug can also be minimized.

The microparticulates prepared in the present invention can be used as injectable preparation or implant pellet for sustained release of drug. Specifically, subcutaneous and intramuscular injection can be enumerated. Additionally, as available formulations thereof, injectable preparations such as injection solution and powder for preparing ready-to-use injection solution, and implant preparations such as pellet can be enumerated. Therefore, the composition of the present invention can further contain excipients, stabilizers, pH regulators and tonicity regulating agents that are conventionally used in preparing pharmaceutical preparations.

### BRIEF EXPLANATION OF DRAWINGS

FIG. 1a is an electron microscopic image on the cross section of polymeric microparticulates prepared in Comparative Example 1.

FIG. 1b is an electron microscopic image on the cross section of polymeric microparticulates prepared according to Examples 1-3.

FIG. 2 illustrates the release profiles of drug depending on the mixing ratio of dichloromethane and acetone in organic solvent including poly(lactic acid) and hydrophobic surfactant (Comparative Example 1: Δ, Example 1: ●, Example 1-1: ▴, Example 1-2: ♦, and Example 1-3: ▪).

FIG. 3 represents the release profiles of drug depending on the mixing ratio of dichloromethane and acetone in organic solvent including poly(lactic acid-co-glycolic acid) and hydrophobic surfactant (Comparative Example 2: ▴ and Example 2: ●).

FIG. 4 represents the release profiles of drug depending on the mixing ratio of poly(lactic acid-co-glycolic acid) and poly(ethylene glycol) (Example 2: ♦, Example 2-1: ▪, Example 2-2: ▴, and Example 2-3: ●).

FIG. 5 shows the release profiles of drug depending on the mixing ratio of dichloromethane and acetone in organic solvent, when chitosan was used instead of sodium hyaluronate as a water-soluble polymer (Comparative Example 3: ●, Example 3: ▴, and Example 3-1: ▪).

FIG. 6 shows the release profiles of drug depending on different viscous internal water phases (Example 2: ▪, Example 3: ▴, and Comparative Example 4: ●).

### BEST MODE FOR CARRYING OUT THE INVENTION

In the below, preferred Examples, Experimental Examples and Preparation Examples of the present invention will be discussed. However, the following specific examples are intended to give easy understanding of the present invention, yet they do not limit the scope of the present invention.

### EXAMPLE 1

Internal water phase was obtained by dispersing sodium alendronate 100 mg in aqueous solution (500 μl) containing sodium hyaluronate (0.75% (w/v) based on water) and poly(ethylene glycol) sorbitan monooleate (20% (w/v) based on water). Polymer solution of organic phase was obtained by dissolving poly(lactic acid) (molecular weight 100,000) 10 parts by weight and sorbitan trioleate 5 parts by weight in a mixture consisting of dichloromethane and acetone (9:1, volume ratio) 100 parts by weight. External continuous phase was obtained by dissolving ethyl acetate 1 part by weight in aqueous solution 99 parts by weight (made by dissolving polyvinylalcohol 0.5 part by weight in distilled water 100 parts by weight).

Internal water phase and organic phase (volume ratio of 1:10) was stirred vigorously to prepare W/O type emulsion. While the external continuous phase was homogeneously dispersed by a homogenizer at 5,000 rpm, W/O type primary emulsion prepared in the above was slowly added thereto in volume ratio of the primary emulsion to the external continuous phase of 1:200 and dispersed by a homogenizer for 5 min to prepare W/O/W type multiple emulsion. After mild stirring for 30 min, organic solvent was removed by filtration, and the remaining product was dried in a vacuum oven for 24 hrs to obtain microparticulates.

### EXAMPLE 1-1

Except that the mixed solvent of dichloromethane and acetone (8:2 ratio) was used as the organic solvent forming organic phase, microparticulates were prepared according to the same method as in Example 1.

### EXAMPLE 1-2

Except that the mixed solvent of dichloromethane and acetone (7:3 ratio) was used as the organic solvent forming organic phase, microparticulates were prepared according to the same method as in Example 1.

### EXAMPLE 1-3

Except that the mixed solvent of dichloromethane and acetone (6:4 ratio) was used as the organic solvent forming organic phase, microparticulates were prepared according to the same method as in Example 1.

### COMPARATIVE EXAMPLE 1

Except that dichloromethane alone was used as the organic solvent forming organic phase, microparticulates were prepared according to the same method as in Example 1.

### EXAMPLE 2

Internal water phase was obtained by dispersing sodium alendronate 100 mg in aqueous solution (500 μl) in which sodium hyaluronate (0.75% (w/v) based on water) and poly(ethylene glycol) sorbitan monooleate (20% (w/v) based on water) were dissolved. Polymer solution of organic phase was obtained by dissolving poly(lactic acid-co-glycolic acid) (molar ratio between lactic acid and glycolic acid=50:50, molecular weight 54,000) 30 parts by weight and sorbitan trioleate 5 parts by weight in mixed solvent consisting of dichloromethane and acetone (7:3 ratio) 100 parts by weight. External continuous phase was obtained by dissolving ethyl acetate 1 part by weight in aqueous solution (99 parts by weight) prepared by dissolving polyvinylalcohol 0.5 part by weight in distilled water 100 parts by weight.

Internal water phase and organic phase (volume ratio of 1:10) was stirred vigorously to prepare W/O type emulsion. While the external continuous phase was homogeneously dispersed by a homogenizer at 5,000 rpm, W/O type primary emulsion prepared in the above was slowly added thereto in volume ratio of the primary emulsion to the external continuous phase of 1:200 and dispersed by a homogenizer for 5 min to prepare W/O/W type multiple emulsion. After mild stirring for about 30 min, organic solvent was removed by filtration and the remaining product was dried in a vacuum oven for 24 hrs to obtain microparticulates.

### EXAMPLE 2-1

Except that poly(lactic acid-co-glycolic acid) (molar ratio of lactic acid-glycolic acid=50:50, molecular weight 54,000) 29.3 parts by weight and poly(ethylene glycol) (molecular weight 3,350) 0.7 part by weight instead of poly(lactic acid-co-glycolic acid) (molar ratio of lactic acid-glycolic acid=50:50, molecular weight 54,000) 30 parts by weight were used, microparticulates were prepared according to the same method as in Example 2.

### EXAMPLE 2-2

Except that poly(lactic acid-co-glycolic acid) (molar ratio of lactic acid-glycolic acid=50:50, molecular weight 54,000) 28.5 parts by weight and poly(ethylene glycol) (molecular weight 3,350) 1.5 parts by weight instead of poly(lactic acid-co-glycolic acid) (molar ratio of lactic acid-glycolic acid=50:50, molecular weight 54,000) 30 parts by weight were used, microparticulates were prepared according to the same method as in Example 2.

### EXAMPLE 2-3

Except that poly(lactic acid-co-glycolic acid) (molar ratio of lactic acid-glycolic acid=50:50, molecular weight 54,000) 27.0 parts by weight, and poly(ethylene glycol) (molecular weight 3,350) 3.0 parts by weight instead of poly(lactic acid-co-glycolic acid) (molar ratio of lactic acid-glycolic acid=50:50, molecular weight 54,000) 30 parts by weight were used, microparticulates were prepared according to the same method as in Example 2.

### COMPARATIVE EXAMPLE 2

Except that dichloromethane alone was used as the organic solvent forming organic phase, microparticulates were prepared according to the same method as in Example 2.

### EXAMPLE 3

Internal water phase was obtained by dispersing sodium alendronate 100 mg in aqueous solution (500 μl) containing lactic acid (1.5 w/v % based on water), chitosan (0.75% based on water), and poly(ethylene glycol) sorbitan monooleate (10% based on water). Polymer solution of organic phase was obtained by dissolving poly(lactic acid-co-glycolic acid) (molar ratio between lactic acid and glycolic acid=50:50, molecular weight 54,000) 30 parts by weight and sorbitan trioleate 5 parts by weight in mixed solvent of dichloromethane and acetone (8:2 ratio) 100 parts by weight. External continuous phase was obtained by dissolving ethyl acetate 1 part by weight in aqueous solution (99 parts by weight) prepared by dissolving polyvinylalcohol 0.5 part by weight in distilled water 100 parts by weight.

Internal water phase and organic phase (volume ratio of 1:10) was stirred vigorously to prepare W/O type emulsion. While external continuous phase was homogeneously dispersed by a homogenizer at 5,000 rpm, W/O type primary emulsion prepared in the above was slowly added thereto in volume ratio of the primary emulsion to the external continuous phase of 1:200 and dispersed by a homogenizer for 5 min to prepare W/O/W type multiple emulsion. After mild stirring for about 30 min, organic solvent was removed by filtration and the remaining product was dried in a vacuum oven for 24 hrs to obtain microparticulates.

### EXAMPLE 3-1

Except that mixed solvent of dichloromethane and acetone (6:4) was used as the organic solvent forming organic phase, microparticulates were prepared according to the same method as in Example 3.

### COMPARATIVE EXAMPLE 3

Except that dichloromethane alone was used as the organic solvent forming organic phase, microparticulates were prepared according to the same method as in Example 3.

### COMPARATIVE EXAMPLE 4

Internal water phase was obtained by dispersing sodium alendronate 200 mg in aqueous solution (500 μl) of gelatin (5 w/v % based on water), and kept at 80° C. Polymer solution of organic phase was obtained by dissolving poly(lactic acid-co-glycolic acid) (molar ratio between lactic acid and glycolic acid=50:50, molecular weight 54,000) 10 parts by weight in dichloromethane 100 parts by weight. External continuous phase was obtained by dissolving ethyl acetate 1 part by weight in aqueous solution (99 parts by weight) prepared by dissolving polyvinylalcohol 0.5 part by weight in distilled water 100 parts by weight.

Internal water phase and organic phase (volume ratio of 1:10) was stirred vigorously at 25° C. to prepare W/O type emulsion. While external continuous phase was homogeneously dispersed by a homogenizer at 5,000 rpm, W/O type primary emulsion prepared in the above was slowly added thereto in volume ratio of the primary emulsion to the external continuous phase of 1:200 and dispersed by a homogenizer for 5 min to prepare W/O/W type multiple emulsion. At this time, temperature of the external continuous phase should be kept at 25° C. After mild stirring for about 30 min, organic solvent was removed by filtration and the remaining product was dried in a vacuum oven for 24 hrs to obtain microparticulates.

### EXPERIMENTAL EXAMPLE 1

### Experiment for Determining Drug Loading (%)

Polymeric microparticulates prepared in the above examples 30 mg were weighed accurately, put in a test tube with cap, dissolved completely in chloroform 5 ml, mixed with distilled water 20 ml and subjected to vigorous stirring for 30 min. The solution was subjected to centrifuge for 5 min at 5000 rpm, and an aliquot of supernatant was taken and concentration of drug was determined by HPLC analysis and based on this, the amount of the drug within microparticulate was calculated, and according to the following formula, loading % of drug encapsulated within polymeric microparticulates was calculated. The result was given in Table 1.
Drug loading (%)=(the weight of the drug within microparticulates/the weight of the microparticulates taken)×100
Drug loading efficiency (%)=(drug loading %/theoretical drug loading %)×100

Herein, theoretical drug loading (%) refers to total weight of the drug used in preparing microparticulates/(total weight of the drug used in preparing microparticulates+total weight of other materials used in preparing microparticulates), and means drug loading (%) obtained based on the assumption that drug used in preparing microparticulates was completely (100%) encapsulated without any loss to external continuous phase during the preparation of microparticulates. In addition, the other materials used in preparing microparticulates refers to the sum of total weight of the materials constituting organic phase such as polyester polymer and hydrophobic surfactant, and total weight of the material constituting internal water phase such as water-soluble polymer and hydrophilic surfactant.

TABLE 1
Loading % and loading efficiency of sodium alendronate

| Samples | Loading (% by weight) | Loading efficiency (%) |
|---|---|---|
| Example 1 | 3.80 | 36.23 |
| Example 1-1 | 5.48 | 52.22 |
| Example 1-2 | 5.92 | 56.44 |
| Example 1-3 | 6.17 | 58.88 |
| Example 2 | 3.76 | 73.52 |
| Example 2-1 | 3.22 | 62.34 |
| Example 2-2 | 2.69 | 51.92 |
| Example 2-3 | 1.59 | 30.67 |
| Comparative example 1 | 2.25 | 21.87 |
| Comparative example 2 | 1.87 | 18.18 |
| Example 3 | 4.09 | 77.02 |
| Example 3-1 | 3.99 | 75.19 |
| Comparative example 3 | 3.46 | 65.11 |
| Comparative example 4 | 4.51 | 38.88 |

As can be seen from the above Table 1, in case dichloromethane was used alone as a organic solvent so that microcoagulation of sodium hyaluronate did not occur, instead of using mixed solvent such as Comparative Examples 1 and 2, drug loading efficiency was only 20%. On the other hand, remarkable increase in drug loading amount was shown in case of microparticulates in which microcoagulation of sodium hyaluronate was derived by using primary organic solvent in combination with secondary organic solvent. In particular, it could be seen based on the results of Examples 1-1, 1-2 and 1-3 that as the content of acetone within the solvent mixture increases, drug loading amount increases. This was interpreted as indicating that as dehydration of aqueous sodium hyaluronate solution increases, physical force of sodium hyaluronate microparticulate itself to retain drug tends to increase, minimizing drug loss during the preparation procedure.

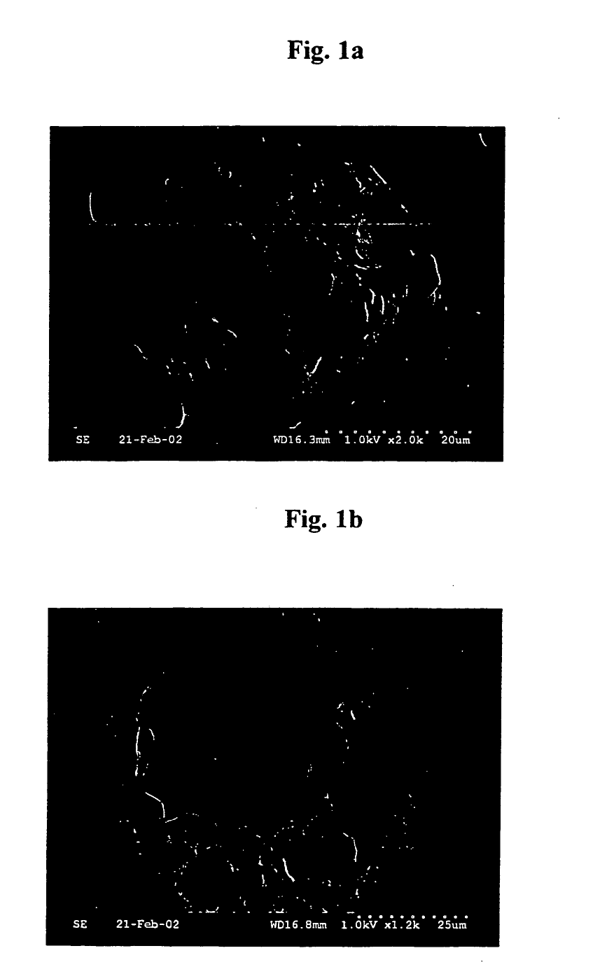

On the other hand, images of the cross section of the prepared polymeric microparticulates, which were taken by differential scanning electron microscope, were shown in FIGS. 1a and 1b. FIG. 1a is a differential scanning electron microscopic image on the cross section of the polymeric microparticulates prepared in Comparative Example 1. Discontinuous internal pores observed in the inside of the microparticulates affect drug loading amount and drug release rate, and as shown by the image, microcoagulation particles of sodium hyaluronate were not formed in the preparation of the microparticulates. As a result, it was supposed that substantial amount of drug was lost to external water phase during the preparation procedure of microparticulates. FIG. 1b is a differential scanning electron microscopic image on the cross section of the polymeric microparticulates prepared in Example 1-3. As shown by the image, the inside of the discontinuous internal pores of the polymeric microparticulates is filled with microcoagulated particles of sodium hyaluronate. Based on this, it could be confirmed that drug was firstly encapsulated within coagulated particles of sodium hyaluronate, thereby minimizing drug loss toward external water phase during the preparation procedure of the microparticulates. Such phenomenon was observed in all Examples, except the case where dichloromethane alone was used in the preparation of microparticulates as in Comparative Examples 1 and 2.

Examples 2, 2-1, 2-2 and 2-3 indicate drug loading amount and loading efficiency according to the increase of poly(ethylene glycol) content, when poly(ethylene glycol) was added to polyester polymer as drug release modifier. As can be seen from Table 1, the addition of poly(ethylene glycol) having features of water solubility and crystallinity causes free influx and outflow of external water phase toward microparticulates during the preparation procedure of microparticulates, resulting in decrease of drug loading amount and loading efficiency.

Examples 3, 3-1 and Comparative Example 3 show drug loading amount and loading efficiency depending on the increase of acetone content within mixed solvent when chitosan was used instead of sodium hyaluronate as internal water phase containing water-soluble polymer. It could be confirmed that in case of chitosan, when a dichloromethane/acetone mixture was used to derive microcoagulation of chitosan, drug loading amount increased, compared to using dichloromethane alone. However, it was confirmed that in case of chitosan, contrary to sodium hyaluronate, no proportional relation exists between the content of acetone within mixed solvent and drug loading efficiency.

### EXPERIMENTAL EXAMPLE 2

### Experiment on In Vitro Release of Drug

To confirm continuous controlled release of hydrophilic and hydrophobic drug from the prepared polymeric microparticulates, drug release experiment was conducted according to the following in vitro condition. That is, the prepared polymeric microparticulates 100 mg was accurately weighed, put in a membrane tube (molecular weight cut-off: 3,500), sealed in both ends, put in a test tube in which pH 7.4 phosphate buffer solution 30 ml was filled, closed with its cap and placed in shaking water bath at 37° C. with 60 times/min speed, allowing sustained release of drug for at least 28 days. An aliquot of 15 ml was taken and the concentration of released drug was determined by HPLC analysis as in Experimental Example 1, and fresh phosphate buffer solution 15 ml was added to the test tube.

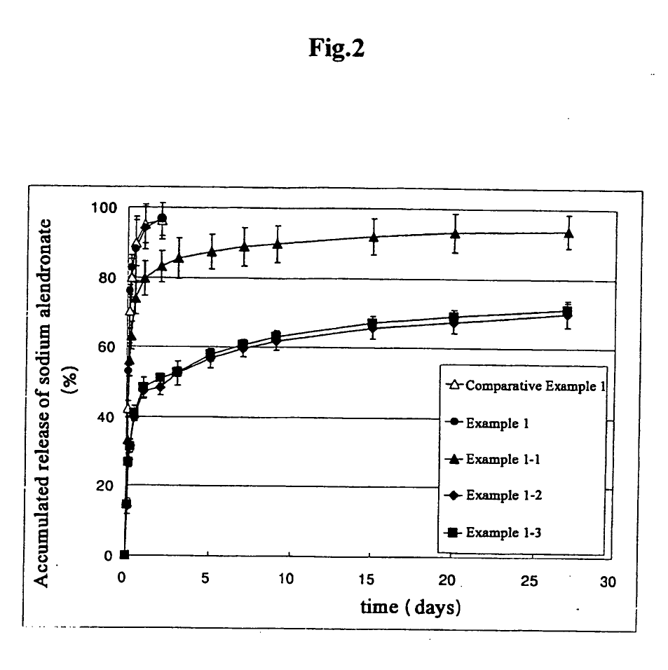

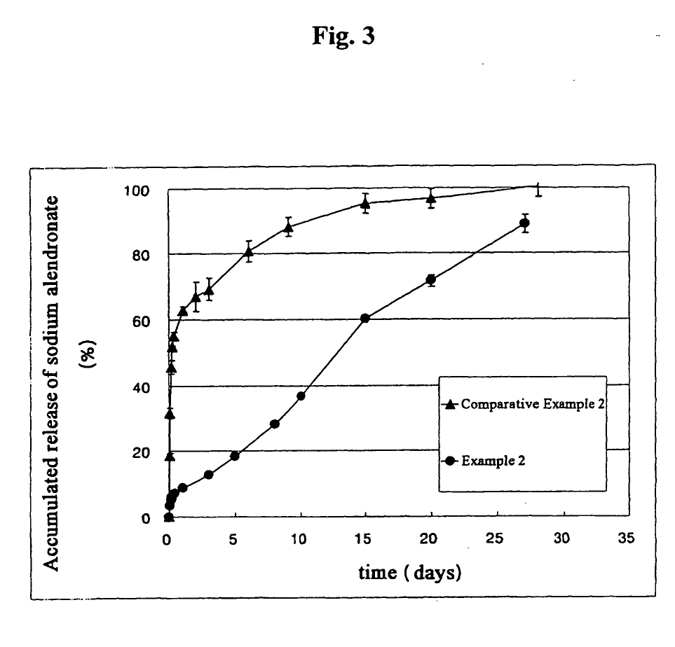

FIGS. 2 and 3 show drug release rate depending on mixing ratio of dichloromethane and acetone in organic solvent, containing poly(lactic acid) (FIG. 2) or poly(lactic acid-co-glycolic acid) (FIG. 3) and hydrophobic surfactant, respectively. Based on the above result, it could be confirmed that as the content of acetone increases, initial release of drug remarkably decreases. It is interpreted as meaning that in case polymeric microparticulates are prepared by adding acetone, since drug is doubly encapsulated in sodium hyaluronate microcoagulation particles and poly(lactic acid) microparticulates, initial release of drug tends to decrease. That is, as the content of acetone within solvent mixture increases, dehydration of aqueous sodium hyaluronate solution of internal water phase increases, leading to increase of coagulation force of microcoagulation particles, and this coagulation force acts as primary controlling factor in drug release.

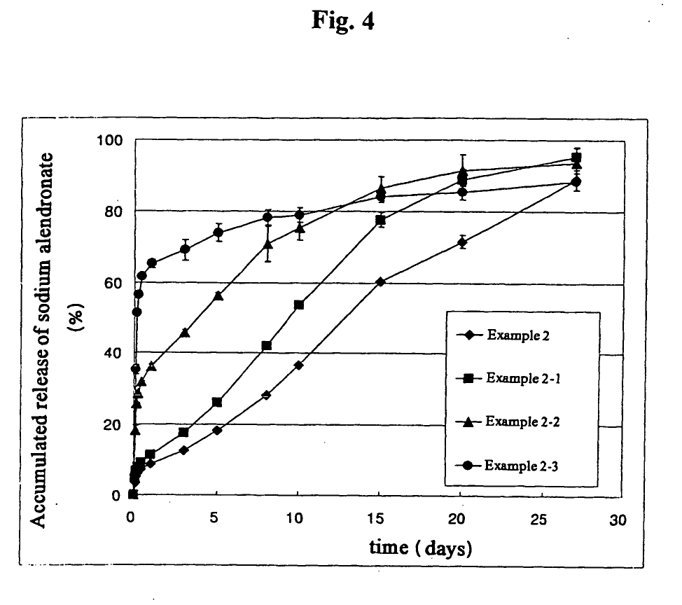

FIG. 4 shows drug release rate depending on mixing ratio between low molecular weight of poly(lactic acid-co-glycolic acid) (mole fraction 50:50) and poly(ethylene glycol). Based on the above result, it could be seen that as the content of poly(ethylene glycol) increases, drug release rate increases. As mentioned above, in case of low molecular weight copolymer in which mole fraction between poly(lactic acid) and poly(glycolic acid) is 50:50, since its physical property is amorphous, rubbery state, the formation of pores and water channels which are main pathway for release of drug encapsulated in microparticulates, is inhibited, leading to lowering of overall release rate of drug. Therefore, in case poly(ethylene glycol) as a drug release modifier is mixed with amorphous polymer, crystalline area is formed within amorphous polymeric microparticulates so that it facilitates the formation of pores and water channels, leading to control of release rate of drug.

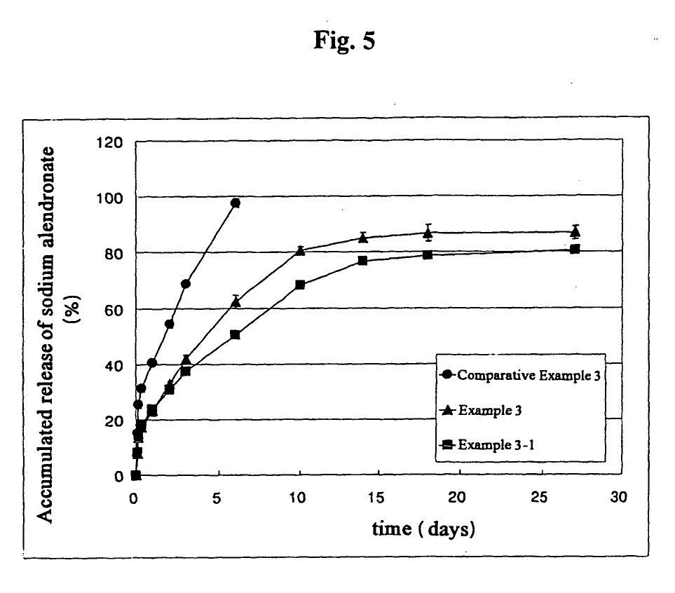

FIG. 5 shows change in early stage release rate of drug when chitosan was used as a water-soluble polymer. It could be seen that as mentioned above, in case chitosan was used (Examples 3 and 3-1), contrary to the case of sodium hyaluronate, no proportional relation exists between the content of acetone within mixed solvent and drug loading efficiency. Yet, it is confirmed that in case a dichloromethane/acetone mixture was used to derive microcoagulation of chitosan, compared to the case of using dichloromethane alone (Comparative Example 3), initial release rate of drug decreased in proportion to the increase of acetone content.

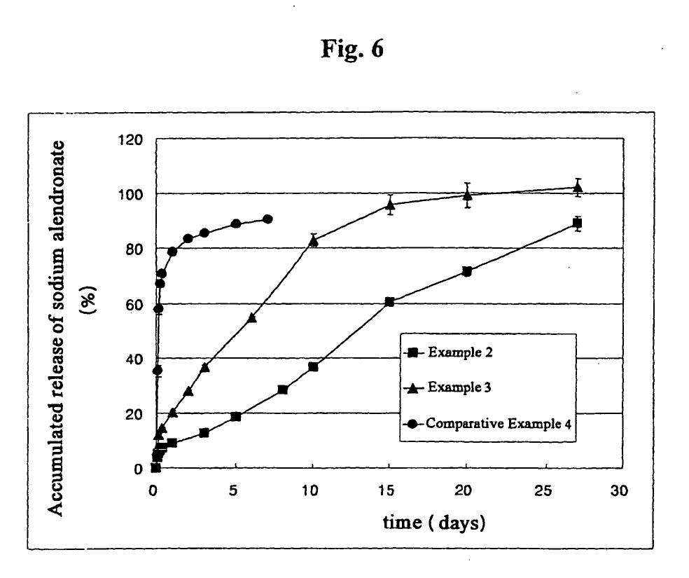

FIG. 6 shows change of early stage release rate of drug according to change of internal water phase having viscosity. It is confirmed that as mentioned above, gelation of gelatin itself (Comparative Example 4) has almost no inhibitory effect on initial burst of drug compared to the derivation of microcoagulation of sodium hyaluronate (Example 2) or chitosan (Example 3). That is, inhibitory effect by gelation of gelatin on initial burst of drug only occurs in limited cases such as protein or peptide drug, and fails to exhibit significant effect on release control of low molecular weight drug such as sodium alendronate, revealing that the technology has no wide applicability.

### PREPARATION EXAMPLE 1

### Injection

Sodium carboxymethylcellulose solution containing sodium chloride and Tween 20 in distilled water for injection was used as an injection vehicle. To reduce pain on injection site, sodium chloride was added to be isotonic, and microspheres were effectively suspended and kept as homogeneous suspension during injection. To allow microsphere particles to stay on injection site, sodium carboxymethylcellulose was used as a thickener for maintaining viscosity of 200 to 400 cps. The injection vehicle was used after sterilization.

The following components were filled to 1.0 ml ample according to conventional method for an injection and sterilized to prepare the injection preparation. At the time of administration, microparticulates composition 50.0 mg prepared under sterilized condition can be administered by mixing with the following injection vehicle composition.

Injection vehicle composition

| | | |
|---|---|---|
| Sodium chloride | 9.0 | mg |
| Sodium carboxymethylcellulose | 30.0 | mg |
| Tween 20 | 1.0 | mg |
| Distilled water for injection | to 1.0 | ml |

### INDUSTRIAL APPLICABILITY

According to the W/O/W multiple emulsion method of the present invention, drug is primarily encapsulated within microcoagulated particles of water-soluble polymer formed in the preparation of primary emulsion, and it is secondarily encapsulated within polyester polymer, thereby improving drug loading amount by minimizing the loss of drug during secondary emulsion process. Further, initial burst of the drug doubly encapsulated within water-soluble polymer and polyester polymer can be minimized, leading to ultimately sustained and prolonged release of the drug.

1. A method for preparing polymeric microparticulates comprising (1) adding secondary organic solvent into primary organic solvent containing biodegradable polymer and hydrophobic surfactant to prepare polymer solution; (2) dissolving and/or dispersing drug(s) in aqueous solution including water-soluble polymer and hydrophilic surfactant, and then adding the solution to the polymer solution prepared in the step (1) to prepare primary emulsion solution (water-in-oil (W/O)), where microcoagulated particles of the water-soluble polymer is formed by dehydration of internal water phase of the primary emulsion solution, leading to encapsulation of the drug into said microparticulates; and (3) dispersing the primary emulsion solution into external continuous phase to solidify the polymeric microparticulates.

2. The method according to claim 1, characterized in that the biodegradable polymer is at least one selected from the group consisting of poly(lactic acid) (PLA), poly(glycolic acid) (PGA), poly(lactic acid-co-glycolic acid) (PLGA) and polycaprolactone (PCL).

3. The method according to claim 1, characterized in that the biodegradable polymer is added to 10 to 60% (w/v) of the organic solvent within the polymer solution.

4. The method according to claim 1, characterized in that, in the step (1), crystalline polymer is further added.

5. The method according to claim 4, characterized in that the crystalline polymer is poly(ethylene glycol) or poly(L-lactic acid).

6. The method according to claim 4, characterized in that mass ratio between the crystalline polymer and the biodegradable polymer is 0.1:99.9 to 20:80.

7. The method according to claim 1, characterized in that said hydrophobic surfactant is at least one selected from the group consisting of fatty acid, olefin, alkyl carbon, silicone, sulfate ester, fatty alcohol sulfate, sulfated fat and oil, sulfonic acid salt, aliphatic sulfonate, alkylaryl sulfonate, ligminsulfonate, phosphoric acid ester, polyoxyethylene, polyglycerol, polyol, imidazoline, alkanolamine, hetamine, sulfomethamine, phosphatide and sorbitan fatty acid ester.

8. The method according to claim 7, characterized in that said hydrophobic surfactant is sorbitan trioleate.

9. The method according to claim 1, characterized in that the hydrophobic surfactant is added to 0.1 to 30% (v/v) of the organic solvent within the polymer solution.

10. The method according to claim 1, characterized in that the primary organic solvent is at least one selected from dichloromethane, chloroform, cyclohexane and ethylacetate.

11. The method according to claim 1, characterized in that the secondary organic solvent is at least one selected from acetone, acetonitrile, dimethylsulfoxide, tetrahydrofuran and dioxane.

12. The method according to claim 1, characterized in that volume ratio between the primary organic solvent and the secondary organic solvent is 95:5 to 50:50.

13. The method according to claim 1, characterized in that the water-soluble polymer is at least one selected from the group consisting of cellulose, hemicellulose, pectin, lignin, starch of storage carbohydrate, chitosan, xanthan gum, alginic acid, pullulan, curdlan, dextran, levan, hyaluronic acid, glucan, collagen and salts thereof.

14. The method according to claim 13, characterized in that the water-soluble polymer is hyaluronic acid or its salt.

15. The method according to claim 1, characterized in that viscosity of the water-soluble polymer in the aqueous solution before dehydration is 300 to 50,000 cps.

16. The method according to claim 1, characterized in that the hydrophilic surfactant is at least one selected from the group consisting of protein surfactant, polyoxyethylene-polyoxypropylene block copolymer and polyoxyethylene sorbitan fatty acid ester.

17. The method according to claim 16, characterized in that the hydrophilic surfactant is polyoxyethylene sorbitan monooleate.

18. The method according to claim 1, characterized in that the hydrophilic surfactant is added to 0.1 to 30% (w/w) of water.

19. The method according to claim 1, characterized in that the drug is bisphosphonates.

20. The method according to claim 1, characterized in that the external continuous phase is aqueous solution of sodium dodecyl sulphate (SDS), cetyltrimethyl ammonium bromide (CTAB), methyl cellulose (MC), gelatin, polyoxyethylene sorbitan monooleate or polyvinyl alcohol (PVA).

21. The method according to claim 1, characterized in that conventional filtration and washing step is further added to the step (3).

22. Polymeric microparticulates obtained by the preparation method according to any one of claims 1 to 21.
